(19) United States
(12) Patent Application Publication
(10) Pub. No.: US 2007/0260066 A1
(43) Pub. Date: Nov. 8, 2007

(54) Process for the Preparation of Telithromycin

(75) Inventors: Suhas Sohani, Vadodara (IN); Mandar Deodhar, Vadudara (IN); Nishant Patel, Vadodara (IN); Manish Patel, Vadodara (IN); Mahesh Davadra, Vadodara (IN); Vinodhamar Kansal, Vadodara (IN)

Correspondence Address: HAMRE, SCHUMANN, MUELLER & LARSON, P.C., P.O. BOX 2902, MINNEAPOLIS, MN, 55402-0902, US

(73) Assignee: Alembic Limited, Vadodara (IN)

(21) Appl. No.: 10/591,889

(22) PCT Filed: Apr. 25, 2005
(86) PCT No.: PCT/IN05/00125

§ 371 Date: Jun. 11, 2007

(30) Foreign Application Priority Data
Apr. 28, 2004 (IN) 491/MUM/2004

(51) Int. Cl. C07D 233/90; C07D 401/02

(52) U.S. Cl. 546/272.7; 548/311.1

(57) ABSTRACT

The present invention relates to the process for the preparation of compounds of formula (I) or its pharmaceutically acceptable salts Formula I The novel compounds (i) 10,11-Anhydro-2′,4″-di-O-benzoyl-12-O-imidazolylcarbonyl-6-O-methylerythromycin A of formula (Xa) Formula (Xa) (ii) 2′,4″-di-O-benzoyl-11-amino-11-N-[4-[4-(3-pyridyl)imidazol-1-yl]butyl]-11-deoxy-6-O-methylerythromycin A 11,12-cyclic carbamate of formula (XIa) Formula (XIa) (iii) 2′-O-benzoyl-11-amino-11-N-[4-[4-(3-pyridyl)imidazol-1-yl]butyl]-11-deoxy-5-O-desosaminyl-6-O-methylerythronolide A 11,12-cyclic carbamate of formula (XIIa) Formula (XIIa) (iv) 2′-benzoyl-11-amino-11-N-[4-[4-(3-pyridyl)imidazol-1-yl]butyl]-11-deoxy-5-O-desosaminyl-6-O-methylerythronolide A 11,12-cyclic carbamate of formula (XIIa) Formula (XIIa) and their use as intermediates in formation of compound of Formula I. Also the process of preparation of compound of formula (XIIIa). Formula (XIIIa)

### FIELD OF THE INVENTION

The present invention relates to the process for the preparation of Telithromycin of formula (I) and its pharmaceutically acceptable salts. 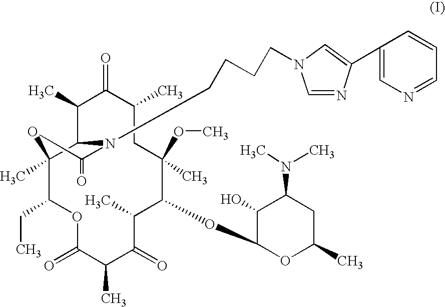
Telithromycin of formula (I) has an antibiotic activity.

### BACKGROUND OF THE INVENTION

Macrolide compounds are known for anti bacterial activity. The rapid development of antibiotic resistance among the major respiratory pathogens has created a serious problem for the effective management of respiratory tract infections. There is a great medical need for new antibiotics to address the problem of antibiotic resistance. Under these circumstances, several novel series of macrolides with a common C-3 ketone group were recently introduced, which are collectively known as ketolides.

Ketolides represent a novel class of macrolide antibiotics that have received much attention recently on account of their excellent activity against resistant organisms. Most ketolides are derivatives of erythromycin, a potent and safe antibiotic widely prescribed for the treatment of respiratory tract infections for more than four decades. Ketolides are 14-membered ring macrolide derivatives characterized by a keto group at the C-3 position [Curr. Med. Chem.—Anti-Infective Agents, 2002, 1, 15-34]. Several Ketolide compounds are under clinical investigation. However, Telithromycin of Formula (I) is the first agent to receive approvable status in this class of drugs.

U.S. Pat. No. 5,635,485 discloses several ketolide compounds, which are prepared by condensing compounds of Formula (II) with amine of formula (III) in a solvent for prolonged hours to yield compound of formula (IV), followed by removal of protecting group Z′ at 2′ position by hydrolysis as shown in Scheme-1. Furthermore, Formula (II) has been prepared by following U.S. Pat. No. 5,527,780. 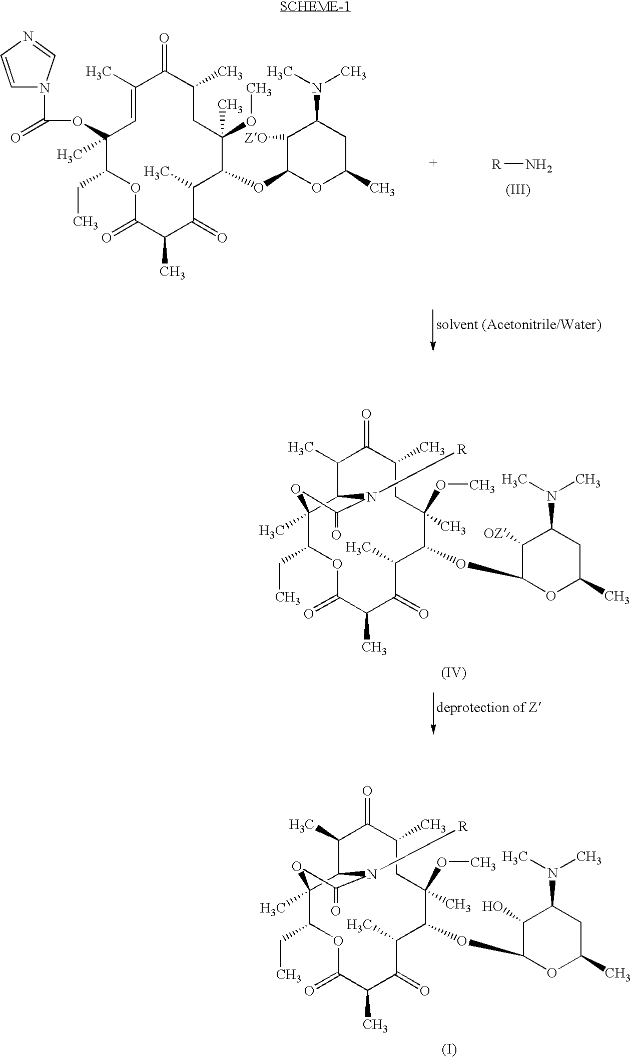
wherein, definition of R and Z′ are as defined in above referred patent.

Accordingly, Telithromycin is prepared by condensing compound of formula (II) with amine of formula (III), where in 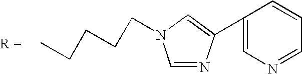
followed by removing the protecting group to yield Telithromycin of formula (I). The preparation of formula II is disclosed in Current Medicinal chemistry, 2001, Vol. 8, 1727-1758. The process described in U.S. Pat. No. 5,635,485 suffers several drawbacks such as
(i) Condensation of formula II with formula III is cumbersome and it is very difficult to remove unreacted reagents and impurities formed during the reaction.
(ii) The isolation and purification of the desired compound of Formula (I) cannot be done without laborious column chromatography, which is not viable at commercial production level.

Current Medicinal Chemistry, 2001, Vol. 8, 1727-1758 also describes the process for the preparation of various ketolides, including Telithromycin in which Clarithromycin (formula V) is reacted with hydrochloric acid to remove cladinose ring at C-3 position (formula VI) followed by selective acetylation of the 2′-hydroxy group in formula VI and selective oxidation of the 3-hydroxy group generated ketolide of formula VII. Further, 11-hydroxy group of compound of formula (VII) is selectively mesylated followed by base induced β-elimination to furnish α,β-unsaturated ketone (formula VIII). The compound of formula (VIII) is further treated with sodium hydride and carbonyldiimidazole to form 12-O-acyl imidazole of formula (II), which upon stereoselective cyclization with (4-(3-pyridinyl)-imidazol-1-yl)-butylamine and subsequent deprotection of the 2′-hydroxy group gives Telithromycin of Formula (I). This process is outlined in following SCHEME-2

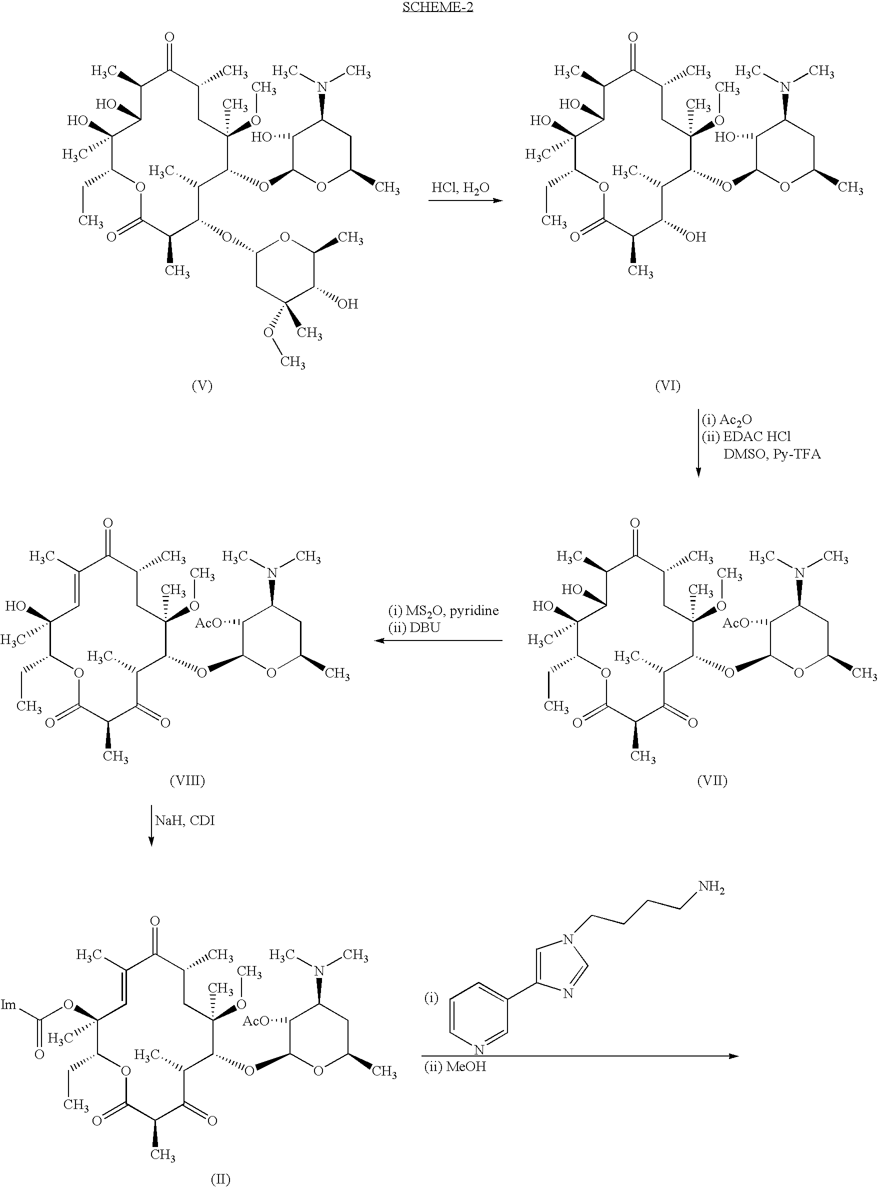

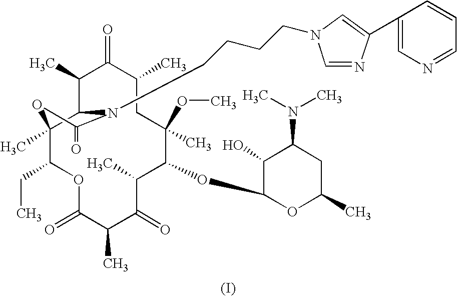

However, this process also consists of several difficulties as explained above and moreover other difficulties such as use of pyrophoric material like NaH, which is hazardous and extremely difficult to handle at the plant scale.

In light of the above difficulties for the preparation of Telithromycin, this process is not suitable for commercial production level.

### OBJECTS OF THE INVENTION

Therefore the basic object of this invention is to provide a process for the preparation of Telithromycin.

Another object of the present invention is to provide a process for the preparation of Telithromycin, which would be high yielding, cost effective, easy to operate at industrial scale and would not involve the use of moisture sensitive, pyrophoric compounds such as sodium hydride.

Another object of the present invention is to provide a process of manufacture of Telithromycin, which lead to the removal of reagents and side products by intermediate crystallization. Thus, isolation of final product enabling good yield and purity, without column chromatography.

A further objective of the invention is to provide a process of manufacture of Telithromycin that would involve selective mild reaction conditions.

A further object of the invention is to provide a process of manufacture of Telithromycin that would be industrially feasible.

Still, another object of the present invention is to provide a novel compound of formula (Xa), (XIa), (XIIa) and (XIIIa), which are useful as an intermediate for the preparation of Telithromycin (I).

### SUMMARY OF THE INVENTION

Present invention provides the process for the preparation of Telithromycin of formula (I) or its pharmaceutically acceptable salts 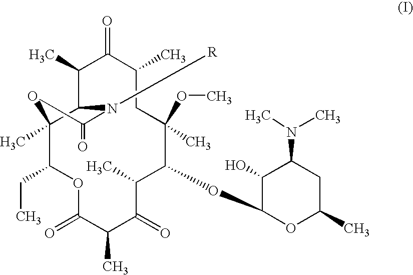
where, R is 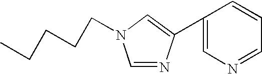
comprising
(a) reacting compound of formula (IX) 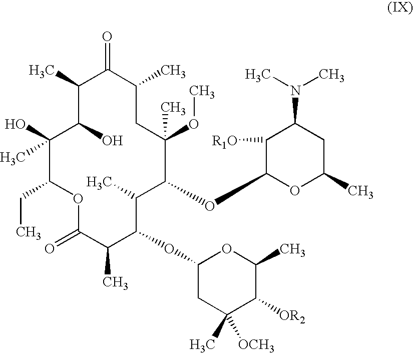
   with carbonyldiimidazole in presence of polar solvent and base to obtain the compound of formula (X) 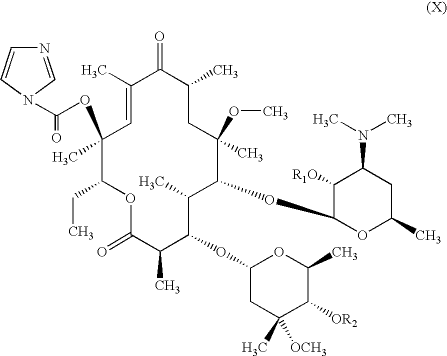
   where R₁ and R₂ are same or different protecting groups represented by 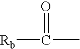
   R_{b} is C₁ to C₁₀ alkyl group or aryl group, preferably R_{b} is C₁-C₄ alkyl group, aryl represents substituted or unsubstituted phenyl group; more preferably R₁ and R₂ are same or different acetyl, benzyl or benzoyl group
(b) condensing the compound of formula (X) with R—NH₂ in suitable polar solvent to give compounds of formula (XI). 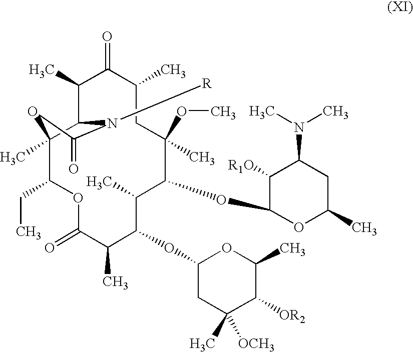
   where R is as defined above and R₁ and R₂ are also same as defined above.
(c) treating the compound of formula (XI) with acid to obtain compound of formula (XII) 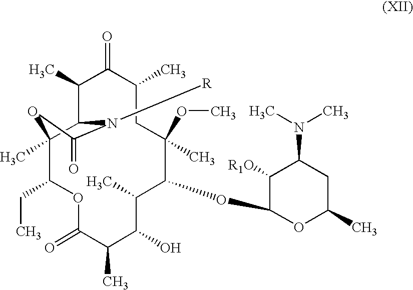
(d) oxidising the resulting compound of formula (XII) in presence of oxidizing agent to give compounds of formula (XIII) 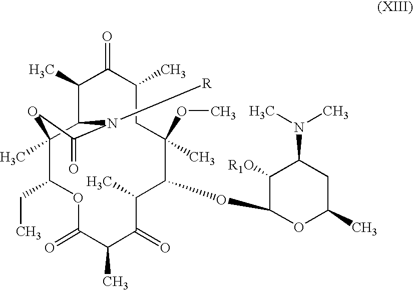
(e) removing the protecting group at 2′ position of formula (XIII) by treating with alcohol to give Telithromycin of formula (I)

The reaction scheme for the preparation of Telithromycin (I) is as shown in Scheme-3 hereunder:

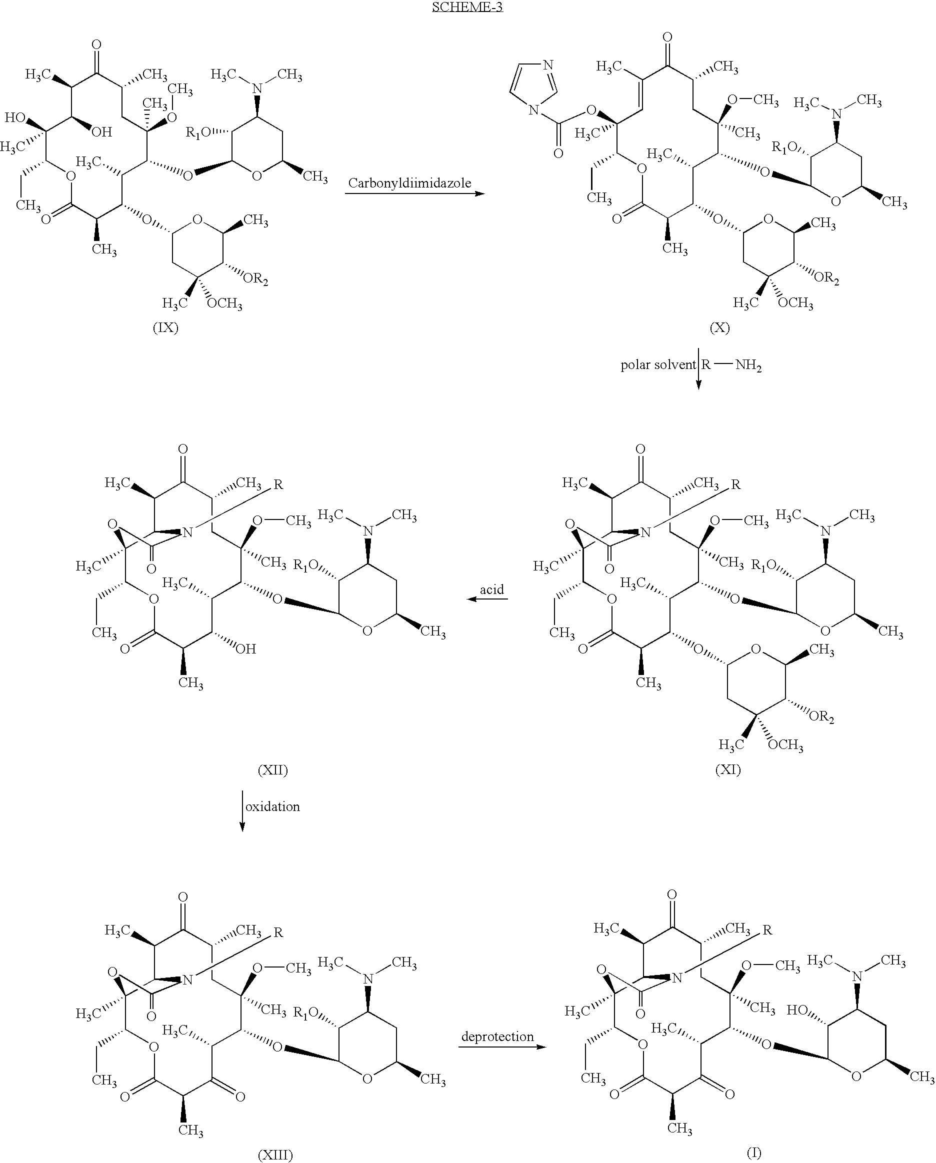

Alternatively, the process for the preparation of Telithromycin of formula (I) or its pharmaceutically acceptable salts, which comprises
(a) reacting compound of formula (IX) 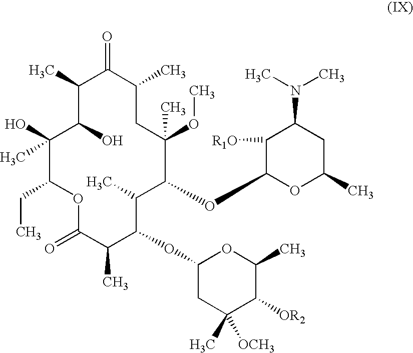
   with carbonylidimidazole in presence of polar solvent and base to obtain the compound of formula (X) 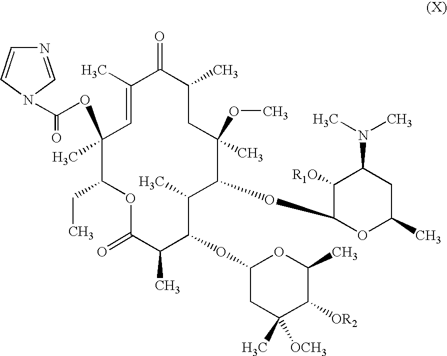
   where R₁ and R₂ are same or different protecting groups represented by 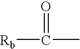
   R_{b} is C₁ to C₁₀ alkyl group or aryl group, preferably R_{b} is C₁-C₄ alkyl group, aryl represents substituted or unsubstituted phenyl group; more preferably R₁ and R₂ are same or different acetyl, benzyl or benzoyl group,
(b) condensing the compound of formula (X) with R—NH₂ in suitable polar solvent to give compounds of formula (XI). 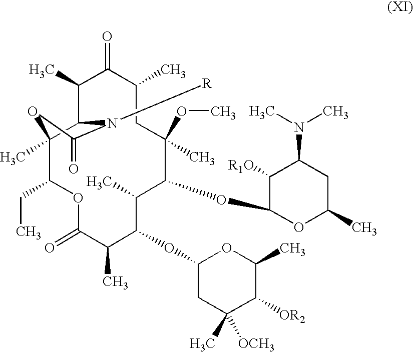
   where R is as defined above and R₁ and R₂ are also same as defined above.
(c) treating the compound of formula (XI) with acid to obtain compound of formula (XII) 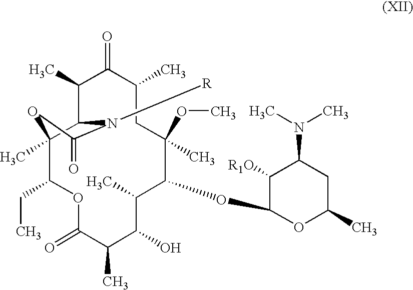
(d) treating compounds of formula (XII) as received from step (c) with alcohol to give compounds of formula (XIV) 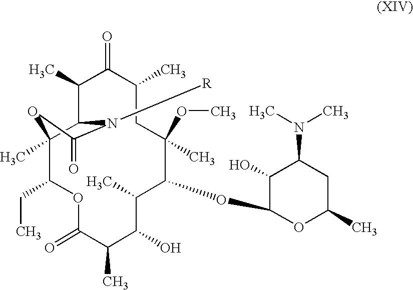
(e) selective oxidization of resulting compounds of formula (XIV) of step (f) in the presence of oxidizing agent to form desired ketolide compound of formula (I)

Optionally the compound of formula XIV may be crystallized using a polar solvent selected from acetone, alcohol, ethyl acetate, preferably acetone.

The reaction scheme followed is as shown in Scheme-4 hereunder:

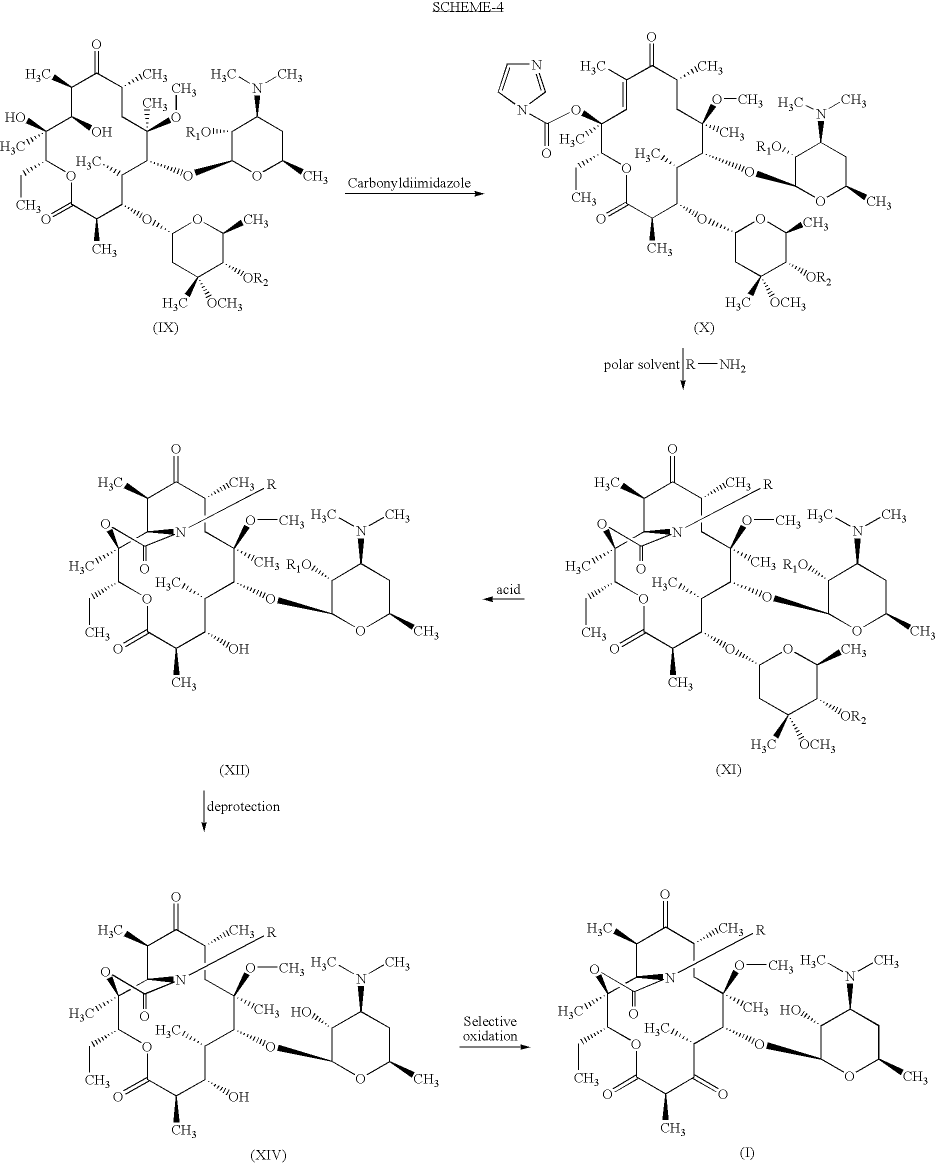

Also, the present invention provides the novel compounds of formula (Xa), (XIa), (XIIa), (XIIIa), where R₁ and/or R₂ is benzoly (Bz) as stated in the above compounds of formula (X), (XI), (XII) and (XIII) respectively. 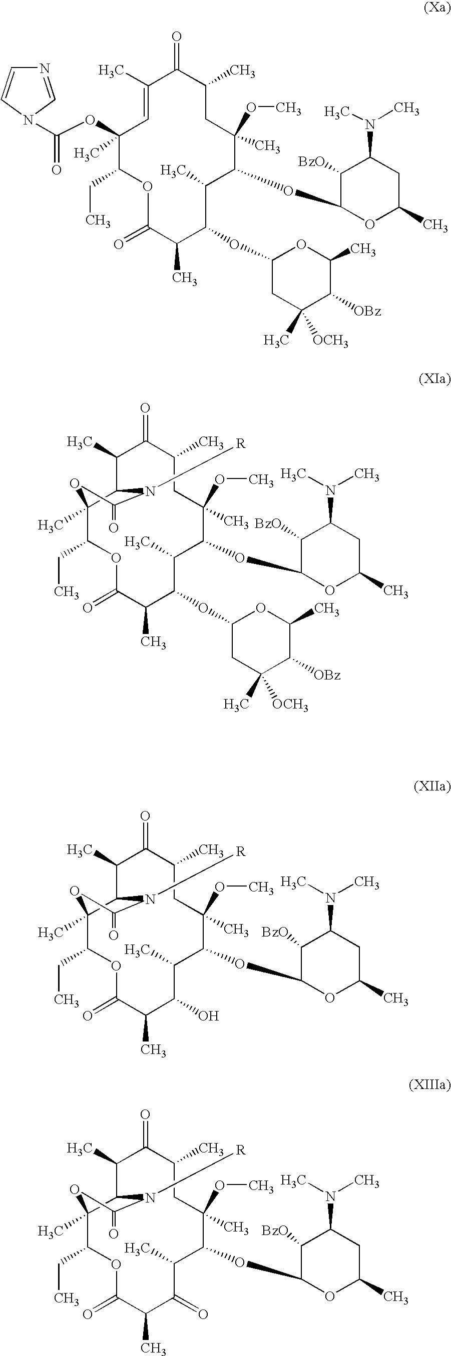
where is R is as defined above.

### DETAILED DESCRIPTION OF THE INVENTION

The polar solvent use in step (a) is selected from dimethylformamide, tetrahydrofuran, acetonitrile and mixtures thereof.

The base used in step (a) is selected from DBU, Triethylamine, diisopropylethylamine.

The polar solvent used in step (b) is a polar aprotic solvent or polar protic solvent. The solvent is selected from the group comprises of methanol, ethanol, isopropanol, n-propanol, n-butanol, iso butyl alcohol, tert-butyl alcohol, methoxyethanol, ethoxyethanol, pentanol, neo-pentyl alcohol, t-pentyl alcohol, cyclohexanol, ethylene glycol, propylene glycol, benzyl alcohol, phenol, glycerol, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethylsulfoxide, propionitrile, ethyl formate, methyl acetate, hexachloroacetone, HMPA, HMPT, acetone, ethyl methyl ketone, ethyl acetate, isopropyl acetate, t-butyl acetate, sulfolane, N,N-dimethylpropionamide, nitromethane, nitrobenzene, teteahydrofuran (THF), dioxane, water, polyethers or mixtures thereof.

Acid referred in step (c) is organic acid or inorganic acid selected from group comprising of hydrochloric acid, hydrobromic acid, sulfuric acid, nitric acid, phosphoric acid, perchloric acid or hydrofluoric acid. The preferred acid is hydrochloric acid. The reaction step (c) is carried out in a solvent selected from water, polar organic solvent such as alcohols selected from methanol, ethanol, isopropanol, n-propanol, tert-butanol, n-butanol or mixture there of.

The said step is carried out at 0° to 70° C. and more preferably at 20 to 60° C.

The oxidation in step (d) is carried out by using the commonly used oxidising reagents such as Corey-Kim oxidation method, Dess-Martins reagent, Pfitzner moffat method or modifications thereof or with dimethyl sulfoxide in presence of oxalyl chloride or phosphorous pentoxide or p-Toluene sulfonyl chloride or acetic anhydride or N-chlrosuccinimide. The oxidation can also be carried out by Manganese or chromium or selenium reagents, tert-amine oxides or by any above oxidant in presence of phase transfer catalyst.

The alcohol referred in step (e) is selected from group comprising of methanol, ethanol, n-propanol, isopropanol, tert-butanol, n-butanol or mixtures there of. The preferred alcohol is methanol.

The reaction step (e) is carried out at a temperature of 0 to 100° C. and preferably at 20 to 70° C. The step (e) can also be carried out in presence of mineral acid selected from HCl or H₂SO₄.

Alternative process for the preparation of Telithromycin, the reaction step (f) is carried out in presence of alcohol to give compounds of formula (XIV). The alcohol in step (f) is selected from group comprising of methanol, ethanol, n-propanol, isopropanol, tert-butanol, n-butanol or mixtures there of. The preferred alcohol is methanol.

The reaction step (f) is carried out at a temperature of 0 to 100° C. and preferably at 20 to 70° C. The step (f) can also be carried out in presence of mineral acid selected from HCl or H₂SO₄.

The compound formula (XIV) is selectively oxidized to give Telithromycin of formula (I). The said oxidation is carried out using Corey-Kim oxidation method, Dess-Martins reagent, Pfitzner moffat method or modifications thereof or with dimethyl sulfoxide in presence of oxalyl chloride or phosphorous pentoxide or p-Toluene sulfonyl chloride or acetic anhydride or N-chlrosuccinimide.

According to another embodiment of the present invention, there is provided a process for the preparation of novel compound of formula (Xa), (XIa), (XIIa), (XIIIa).

The process for the preparation of compound of formula (XIIIa), which comprises
(a′) reacting 2′,4″-di-O-benzoyl-6-O-methylerythromycin A compound of formula (IXa) 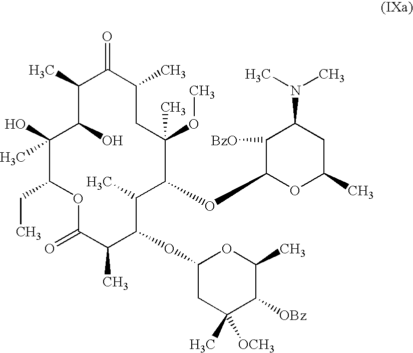
with carbonyldiimidazole in presence of a polar solvent and base to obtain 10,11-anhydro-2′,4″-di-O-benzoyl-12-O-imidazolylcarbonyl-6-O-methylerythromycin A (Xa) 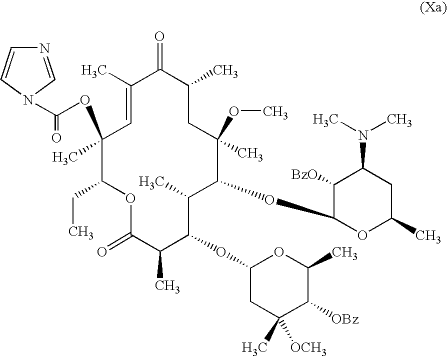
(b′) condensing the compounds of formula (Xa) with R—NH₂ in a suitable polar solvent in presence of base to obtain 2′,4″-di-O-benzoyl-11-amino-11-N-[4-[4-(3-pyridyl)imidazol-1-yl]butyl]-11-deoxy-6-O-methylerythromycin A 11,12-cyclic carbamate of formula (XIa) 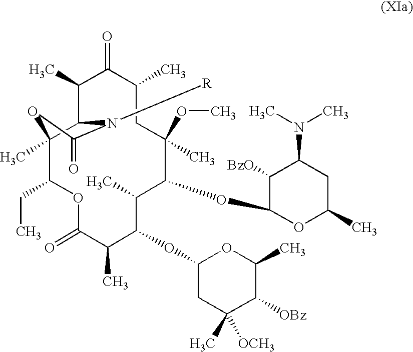
(c′) treating the obtained compound formula (XIa) with an acid to give 2′-O-benzoyl-11-amino-11-N-[4-[4-(3-pyridyl)imidazol-1-yl]butyl]-11-deoxy-5-O-desosaminyl-6-O-methylerythronolide A 11,12-cyclic carbamate of formula (XIIa) 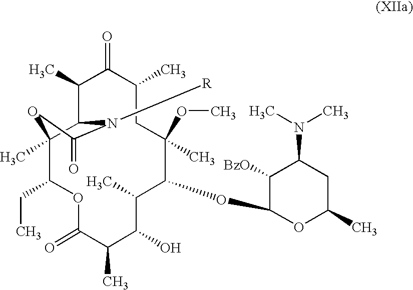
(d′) oxidizing the resulting compounds of formula (XIIa) in presence of oxidizing agent to obtain the compound of formula (XIIIa)

The compound of formula (XIIIa) obtained by above process can be converted to Telithromycin (I) or its pharmaceutically acceptable salts by treating it with alcohols.

The polar solvent used in step (a′) is selected from dimethylformamide, tetrahydrofuran, acetonitrile and mixtures thereof.

The reaction step (a′) is carried out in presence of base selected from DBU, triethylamine, diisopropylethylamine.

Polar solvent used in step (b′) is selected from group comprising of methanol, ethanol, isopropanol, n-propanol, n-butanol, iso butyl alcohol, tert-butyl alcohol, methoxyethanol, ethoxyethanol, pentanol, neo-pentyl alcohol, tert-pentyl alcohol, cyclohexanol, ethylene glycol, propylene glycol, benzyl alcohol, phenol, glycerol, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethylsulfoxide, propionitrile, ethyl formate, methyl acetate, hexachloroacetone, HMPA, HMPT, acetone, ethyl methyl ketone, ethyl acetate, isopropyl acetate, t-butyl acetate, sulfolane, N,N-dimethylpropionamide, nitromethane, nitrobenzene, tetrahydrofuran (THF), dioxane, water, polyethers or mixtures thereof. The preferred solvent is dimethylformamide or acetonitrile.

The reaction step (b′) is carried out in presence of base selected from DBU, triethylamine, diisopropylethylamine. The said step is preferably carried out at a temperature 30° C. to 60° C.

Acid referred in step (c′) is organic acid or inorganic acid selected from group comprising of hydrochloric acid, hydrobromic acid, sulfuric acid, nitric acid, phosphoric acid, perchloric acid or hydrofluoric acid. The preferred acid is hydrochloric acid. The reaction step (c′) is carried out in a solvent selected from water, polar organic solvent such as alcohols selected from methanol, ethanol, isopropanol, n-propanol, tert-butanol, n-butanol or mixture there of.

The said step is carried out at 0 to 70° C. and more preferably at 20 to 60° C.

The oxidation in step (d′) is carried out by using the commonly used oxidising reagents such as Corey-Kim oxidation method, Dess-Martin reagent, Pfitzner Moffat method or modifications thereof or with dimethyl sulfoxide in presence of oxalyl chloride or phosphorous pentoxide or p-Toluene sulfonyl chloride or acetic anhydride or N-chlorosuccinimide. The oxidation can also be carried out by Manganese or chromium or selenium reagents, tert-amine oxides or by any above oxidant in presence of phase transfer catalyst.

Another embodiment of the present invention, the process for the preparation of Telithromycin of formula (I) comprises
(i) reacting 2′4″-di-O-acetyl-6-O-methylerythromycin A (obtained as indicated in example 1(1) of U.S. Pat. No. 5,591,837) with carbonyldiimidazole in presence of polar solvent and base to give 10,11-Anhydro-2′4″-di-O-acetyl-12-O-imidazolyl carbonyl-6-O-methylerythromycin A, the polar solvent being selected from Dimethylformamide, Tetrahydrofuran, Acetonitrile and mixtures thereof and the base selected from DBU, Triethylamine, diisopropylethylamine.
(ii) condensing 10,11-anhydro-2′,4″-di-O-acetyl-12-O-imidazolyl carbonyl-6-O-methylerythromycin A with 4-[4-(3-pyridyl)imidazol-1-yl]butaneamine in a polar solvent at 50 to 120° C. to give 2′,4″-di-O-acetyl-11-amino-11-N-[4-[4-(3-pyridyl)imidazol-1-yl]butyl]-11-deoxy-6-O-methylerythromycin A 11,12-cyclic carbamate, said polar solvent is polar aprotic solvent or polar protic solvent;
(iii) reacting 2′,4″-di-O-acetyl-11-amino-11-N-[4-[4-(3-pyridyl)imidazol-1-yl]butyl]-11-deoxy-6-O-methylerythromycin A 11,12-cyclic carbamate with acid at 0° C. to 100° C. by removal cladinose ring at C-3 position to obtain 2′-O-acetyl-11-amino-11-N-[4-[4-(3-pyridyl)imidazol-1-yl]butyl]-11-deoxy-5-O-desosaminyl-6-O-methylerythronolide A 11,12-cyclic carbamate.
(iv) Further, 2′-O-acetyl-11-amino-11-N-[4-[4-(3-pyridyl)imidazol-1-yl]butyl]-11-deoxy-5-O-desosaminyl-6-O-methylerythronolide A 11,12-cyclic carbamate is oxidized at C-3 position to give 2′-O-acetyl-11-amino-11-N-[4-[4-(3-pyridyl)imidazol-1-yl]butyl]-11-deoxy-3-oxo-5-O-desosaminyl-6-O-methylerythronolide A 11,12-cyclic carbamate;
(v) 2′-O-acetyl-11-amino-11-N-[4-[4-(3-pyridyl)imidazol-1-yl]butyl]-11-deoxy-3-oxo-5-O-desosaminyl-6-O-methylerythronolide A 11,12-cyclic carbamate is further treated with alcohols to remove protecting group at 2′ position to give Telithromycin of formula (I).

In step (ii) of the process the polar solvents is selected form the group comprises of methanol, ethanol, n-propanol, isopropanol, n-butanol, isobutyl alcohol, tert-butyl alcohol, methoxyethanol, ethoxyethanol, pentanol, neo-pentyl alcohol, t-pentyl alcohol, cyclohexanol, ethylene glycol, propylene glycol, benzyl alcohol, phenol, glycerol, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethylsulfoxide, propionitrile, ethyl formate, methyl acetate, hexachloroacetone, HMPA, HMPT, acetone, ethyl methyl ketone, ethyl acetate, isopropyl acetate, t-butyl acetate, sulfolane, N,N-dimethylpropionamide, nitromethane, nitrobenzene, tetrahydrofuran (THF), dioxane, polyethers or water or mixtures thereof.

The preferred polar solvent is dimethylformamide (DMF) and acetonitrile.

The most preferred solvent is dimethylformamide.

The reaction step is carried out at 5 to 120° C. Preferably step (i) can be carried out at 30 to 60° C. The reaction also can be carried out in water or the mixture of water and organic solvents (as mentioned above).

The ratio of substrate to amine is 1:3 mole and the preferably ratio is 1:2 mole.

In step (iii) of the process the acid is selected from organic acid or inorganic acid or mixtures thereof. Inorganic acid can be mineral acid selected from hydrochloric acid, hydrobromic acid, sulfuric acid, nitric acid, phosphoric acid and perchloric acid, hydrofluoric acid. The acid is preferably hydrochloric acid.

The solvent is selected from the group comprising water or polar organic solvents like alcohols or mixtures thereof. The preferred solvents can be water, methanol, ethanol, iso propanol, n-butanol, tert-butanol or mixtures thereof.

The reaction step is carried out at 0 to 100° C. and more preferably 20° C. to 60° C. for 6 to 48 hrs.

In step (iv) of the process the oxidation can be carried out by way of Corey-Kim oxidation method, Dess-Martins reagent, Pfitzner Moffat method or modifications thereof or with dimethyl sulfoxide in presence of oxalyl chloride or phosphorous pentoxide or p-Toluene sulfonyl chloride or acetic anhydride or N-chlorosuccinimide. The oxidation can also be carried out by Manganese or chromium or selenium reagents, tert-amine oxides or any above oxidant in presence of phase transfer catalyst.

In step (v) of the process the alcohol is selected from the group comprising of methanol, ethanol, n-propanol, isopropanol, n-butanol, tert-butanol or mixtures there of or with water at 0° C. to 100° C. to give desired ketolide compounds of formula (I).

Alternatively, pure and commercially viable process for the preparation of Telithromycin is by carrying out first deprotection of 2′-O-acetyl-11-amino-11-N-[4-[4-(3-pyridyl)imidazol-1-yl]butyl]-11-deoxy-5-O-desosaminyl-6-O-methylerythronolide A 11,12-cyclic carbamate as obtained in step (iii) purification and then oxidation of the resultant compound.

The detail process is described as below:

(vi) 2′-O-acetyl-11-amino-11-N-[4-[4-(3-pyridyl)imidazol-1-yl]butyl]-11-deoxy-5-O-desosaminyl-6-O-methylerythronolide A 11,12-cyclic carbamate is treated with an alcohol at 0 to 70° C. or with water at 0° to 100° C. to remove acetyl protecting group and form 2′-hydroxy-11-amino-11-N-[4-[4-(3-pyridyl)imidazol-1-yl]butyl]-11-deoxy-5-O-desosaminyl-6-O-methylerythronolide A 11,12-cyclic carbamate or with water at 0° C. to 100° C. to give desired compound of formula (XIV) the compound of formula (XIV) is crystallised by using polar solvent.

(vii) 2′-hydroxy-11-amino-11-N-[4-[4-(3-pyridyl)imidazol-1-yl]butyl]-11-deoxy-5-O-desosaminyl-6-O-methylerythronolide A 11,12-cyclic carbamate is selectively oxidized at C-3 position to form Telithromycin of formula (I).

In step (vi) the alcohol is selected from the group comprising of methanol, ethanol, n-propanol, isopropanol, n-butanol, tert-butanol or mixtures thereof. In the said step the polar solvent is selected from acetone or alcohol or ethyl acetate or mixture thereof. The solvent used for the crystallisation of formula (XIV) is preferably acetone.

In step (vii) the oxidation is carried out by way of Corey-Kim oxidation method, Dess-Martin reagent, Pfitzner Moffat method or modifications thereof or with dimethyl sulfoxide in presence of oxalyl chloride or phosphorous pentoxide or p-Toluene sulfonyl chloride or acetic anhydride. The oxidation can also be carried out by manganese or chromium or selenium reagents, tert-amine oxides or any above oxidant in presence of phase transfer catalyst.

The process of the present invention is described by the following examples, which are illustrative only and should not be construed so as to limit the scope of the invention in any manner.

### EXAMPLES

### Example 1

### Preparation of 10,11-Anhydro-2′4″-di-O-acetyl-12-O-imidazolyl carbonyl-6-O-methylerythromycin A

Mix 10 gm of 2′,4″-di-O-acetyl-6-O-methylerythromycin A, 10 gm of carbonyldiimidazole, 40 ml dimethylformamide and 4 ml DBU at room temperature. The solution was cleared. The clear solution was stirred for 3 hrs. The reaction mixture was quenched with water (400 ml). The solid was filtered and washed with water. The wet solid was dissolved in dichloromethane and the organic layer was separated. The solvent was removed under vacuum. Add diisopropylether (40 ml) and the reaction mixture was stirred for half an hour. The solid was filtered and washed with diisopropylether (2×5 ml). The solid was dried at room temperature to give 9.0 gm of 10,11-Anhydro-2′4″-di-O-acetyl-12-O-imidazolylcarbonyl-6-O-methyl erythromycin A.

### Example 2

### Preparation of 2′,4″-di-O-acetyl-11-amino-11-N-[4-[4-(3-pyridyl)imidazol-1-yl]butyl]-11-deoxy-6-O-methylerythromycin A 11,12-cyclic carbamate

20 gm of 10,11-Anhydro-2′,4″-di-O-acetyl-12-O-imidazolylcarbonyl-6-O-methyl erythromycin A was added in 9.6 gm of 4-[4-(3-pyridyl)imidazol-1-yl]butanamine and 100 ml Dimethyl formamide and stirred at 50° C. for 18 hours. The reaction mixture was then diluted with water and stirred for 30 min. The precipitated solid was filtered and washed with water. Further, it was dried at 50° C. under vacuum to give 18 gm of 2′,4″-di-O-acetyl-11-amino-11-N-[4-[4-(3-pyridyl)imidazol-1-yl]butyl]-11-deoxy-6-O-methylerythromycin A 11,12-cyclic carbamate.

### Example 3

### Preparation of 2′-O-acetyl-11-amino-11-N-[4-[4-(3-pyridyl) imidazol-1-yl]butyl]-11-deoxy-5-O-desosaminyl-6-O-methylerythronolide A 11,12-cyclic carbamate

23 g of 2′,4″-di-O-acetyl-11-amino-11-N-[4-[4-(3-pyridyl)imidazol-1-yl]butyl]-11-deoxy-6-O-methylerythromycin A 11,12-cyclic carbamate was dissolved in solution of 23 ml concentrated hydrochloric acid and 230 ml water. The mixture was stirred at ambient temperature for 12 hours. The reaction mixture was then basified with sodium hydroxide when a white solid was obtained. The solid was filtered and washed with water. Drying at ambient temperature afforded 16 gm of 2′-O-acetyl-11-amino-11-N-[4-[4-(3-pyridyl)imidazol-1-yl]butyl]-11-deoxy-5-O-desosaminyl-6-O-methylerythronolide A 11,12-cyclic carbamate. The product can be used in the next step without further purification.

### Example 4

### Preparation of 2′-O-acetyl-11-amino-11-N-[4-[4-(3-pyridyl) imidazol-1-yl]butyl]-11-deoxy-3-oxo-5-O-desosaminyl-6-O-methylerythronolide A 11,12-cyclic carbamate

15 g of 2′-O-acetyl-11-amino-11-N-[4-[4-(3-pyridyl)imidazol-1-yl]butyl]-11-deoxy-5-O-desosaminyl-6-O-methylerythronolide A 11,12-cyclic carbamate was dissolved in 150 ml dichloromethane and 22.5 g of Dess Martin reagent was added in one lot. The mixture was stirred at ambient temperature for 1 hour. Further, added the mixture of 260 ml saturated sodium bicarbonate solution and saturated sodium thiosulfate solution and stirred the mixture for 20 min. Filtered off the formed solid precipitate and separated the organic layer. Washed the organic layer with water, dried over sodium sulfate and distilled off the solvent under vacuum to give 13 gm of 2′-O-acetyl-11-amino-11-N-[4-[4-(3-pyridyl)imidazol-1-yl]butyl]-11-deoxy-3-oxo-5-O-desosaminyl-6-O-methylerythronolide A 11,12-cyclic carbamate.

### Example 5

### Preparation of 2′-O-acetyl-11-amino-11-N-[4-[4-(3-pyridyl) imidazol-1-yl]butyl]-11-deoxy-3-oxo-5-O-desosaminyl-6-O-methylerythronolide A 11,12-cyclic carbamate

10 g of 2′-O-acetyl-11-amino-11-N-[4-[4-(3-pyridyl)imidazol-1-yl]butyl]-11-deoxy-5-O-desosaminyl-6-O-methylerythronolide A 11,12-cyclic carbamate was dissolved in 100 ml dichloromethane. Dimethyl sulfoxide (16.6 ml), Cyclohexyl dimethyl amino propyl carbodimide Hydro chloride (25.0 g) and Pyridine HCl (12.1 gm) was added in one lot. The mixture was stirred at ambient temperature (20-30° C.) for 6 hour. Further, 500 ml water was added and stirred the mixture for 30 min. The organic layer was separated and washed with water, dried over sodium sulfate and distilled off the solvent under vacuum to give 7.05 gm of 2′-O-acetyl-11-amino-11-N-[4-[4-(3-pyridyl)imidazol-1-yl]butyl]-11-deoxy-3-oxo-5-O-desosaminyl-6-O-methylerythronolide A 11,12-cyclic carbamate.

### Example 6

### Preparation of 2′-O-acetyl-11-amino-11-N-[4-[4-(3-pyridyl)imidazol-1-yl]butyl]-11-deoxy-3-oxo-5-O-desosaminyl-6-O-methylerythronolide A 11,12-cyclic carbamate

N-Chloro succinimide (4.68 gm) was charged to the reaction vessel under nitrogen atmosphere and Dichloromethane (200 ml) was added slowly. The reaction masses was cooled to 0° C., Dimethyl sulfide (3.5 ml) was added slowly and continue the stirring at same temperature for half an hour. The reaction mass was cooled to −25° C. and 2′-O-acetyl-11-amino-11-N-[4-[4-(3-pyridyl)imidazol-1-yl]butyl]-11-deoxy-5-O-desosaminyl-6-O-methylerythronolide A 11,12-cyclic carbamate (10 g) dissolved in 50 ml dichloromethane was added slowly. The mixture was stirred at −25° C. temperature for 2 hour. At the same temperature Diisopropyletheyl amine (0.6 ml) was added and the reaction mixture was stirred for an hour. Water (500 ml) was added and stirred the mixture for 30 min. The organic layer was separated and washed with water, dried over sodium sulfate and distilled off the solvent under vacuum to give 7.5 gm of 2′-O-acetyl-11-amino-1-N-[4-[4-(3-pyridyl)imidazol-1-yl]butyl]-11-deoxy-3-oxo-5-O-desosaminyl-6-O-methylerythronolide A 11,12-cyclic carbamate.

### Example 7

### 11-Amino-11-N-[4-[4-(3-pyridyl)imidazol-1-yl]butyl-11-deoxy-3-oxo-5-O-desosaminyl-6-O-methylerythronolide A 11,12-cyclic carbamate [Telithromycin] (I)

A solution for 10 g of 2′-O-acetyl-11-amino-11-N-[4-[4-(3-pyridyl)imidazol-1-yl]butyl]-11-deoxy-3-oxo-5-O-desosaminyl-6-O-methylerythronolide A 11,12-cyclic carbamate in 100 ml methanol was stirred at ambient temperature for 16 hours. Further, solvent was distilled off under vacuum and stirring remain solid with 50 ml diisoproyl ether to gave 7 gm of desired compound 11-Amino-11-N-[4-[4-(3-pyridyl)imidazol-1-yl]butyl-11-deoxy-3-oxo-5-O-desosaminyl-6-O-methylerythronolide A 11,12-cyclic carbamate [Telithromycin] (I).

### Example 8

### 11-Amino-11-N-[4-[4-(3-pyridyl)imidazol-1-yl]butyl-11-deoxy-3-oxo-5-O-desosaminyl-6-O-methylerythronolide A 11,12-cyclic carbamate [Telithromycin] (I)

A solution for 10 g of 2′-O-acetyl-11-amino-11-N-[4-[4-(3-pyridyl)imidazol-1-yl]butyl]-11-deoxy-3-oxo-5-O-desosaminyl-6-O-methylerythronolide A 11,12-cyclic carbamate in 100 ml Isopropanol was stirred at ambient temperature for 24 hours. Further, solvent was distilled off under vacuum and equilibrating remain solid with 50 ml diisoproyl ether to gave 7 gm of desired compound 11-Amino-11-N-[4-[4-(3-pyridyl) imidazol-1-yl]butyl-11-deoxy-3-oxo-5-O-desosaminyl-6-O-methylerythronolide A 11,12-cyclic carbamate [Telithromycin] (I).

### Example 9

### 2′-hydroxy-11-amino-11-N-[4-[4-(3-pyridyl)imidazol-1-yl]butyl]-11-deoxy-5-O-desosaminyl-6-O-methylerythronolide A 11,12-cyclic carbamate

10 g of 2′-O-acetyl-11-amino-11-N-[4-[4-(3-pyridyl)imidazol-1-yl]butyl]-11-deoxy-5-O-desosaminyl-6-O-methylerythronolide A 11,12-cyclic carbamate in 100 ml methanol was stirred at reflux temperature for 6 hours. The solvent was then distilled off under vacuum to give crude product as white foam. Then, the crude product was purified by refluxing in 20 ml of acetone followed by 1 hour stirring at 10° C. Filtered off the solution and washed the solid and with 2×5 ml of chilled acetone to gave 8.0 gm 2′-hydroxy-11-amino-11-N-[4-[4-(3-pyridyl)imidazol-1-yl]butyl]-11-deoxy-5-O-desosaminyl-6-O-methylerythronolide A 11,12-cyclic carbamate

### Example 10

### 2′-hydroxy-11-amino-11-N-[4-[4-(3-pyridyl)imidazol-1-yl]butyl]-11-deoxy-5-O-desosaminyl-6-O-methylerythronolide A 11,12-cyclic carbamate

10 g of 2′-O-acetyl-11-amino-11-N-[4-[4-(3-pyridyl)imidazol-1-yl]butyl]-11-deoxy-5-O-desosaminyl-6-O-methylerythronolide A 11,12-cyclic carbamate in 100 ml Isoproapnol was stirred at reflux temperature for 18 hours. The solvent was then distilled off under vacuum to give crude product as white foam. Then, the crude product was purified by refluxing in 20 ml of acetone followed by 1 hour stirring at 10° C. Filtered off the solution and washed the solid and with 2×5 ml of chilled acetone to gave 8.0 gm 2′-hydroxy-1-amino-11-N-[4-[4-(3-pyridyl) imidazol-1-yl]butyl]-11-deoxy-5-O-desosaminyl-6-O-methylerythronolide A 11,12-cyclic carbamate

### Example 11

### 11-Amino-11-N-[4-[4-(3-pyridyl)imidazol-1-yl]butyl-11-deoxy-3-oxo-5-O-desosaminyl-6-O-methylerythronolide A 11,12-cyclic carbamate [Telithromycin] (Formula I)

10 g of 2′-hydroxy-11-amino-11-N-[4-[4-(3-pyridyl)imidazol-1-yl]butyl]-11-deoxy-5-O-desosaminyl-6-O-methylerythronolide A 11,12-cyclic carbamate was dissolved in 200 ml dichloromethane and 15 g of Dess Martin reagent was added in one lot. The mixture was stirred at ambient temperature for 30 min. Further, 260 ml of saturated sodium bicarbonate solution added and stirred the mixture for 30 min. Filtered off the solid precipitate and separated the organic layer. Washed the organic layer with water, dried over sodium sulfate and distilled off the solvent under vacuum to give solids. Further, it was stirred with 40 ml of diisoproyl ether and filtered off and dried to give 9 gm 11-Amino-11-N-[4-[4-(3-pyridyl)imidazol-1-yl]butyl-11-deoxy-3-oxo-5-O-desosaminyl-6-O-methylerythronolide A 11,12-cyclic carbamate [Telithromycin](I).

### Example 12

### 11-Amino-11-N-[4-[4-(3-pyridyl)imidazol-1-yl]butyl-11-deoxy-3-oxo-5-O-desosaminyl-6-O-methylerythronolide A 11,12-cyclic carbamate [Telithromycin] (Formula I)

10 g of 2′-hydroxy-11-amino-11-N-[4-[4-(3-pyridyl)imidazol-1-yl]butyl]-11-deoxy-5-O-desosaminyl-6-O-methylerythronolide A 11,12-cyclic carbamate was dissolved in 100 ml dichloromethane. Dimethyl sulfoxide (16.6 ml), Cyclohexyl dimethyl amino propyl carbodimide Hydro chloride (25.0 g) and Pyridine HCl (12.1 gm) was added in one lot. The mixture was stirred at ambient temperature (20-30° C.) for 6 hour. Further, 500 ml water was added and stirred the mixture for 30 min. The organic layer was separated and washed with water, dried over sodium sulfate and distilled off the solvent under vacuum to give 8.05 gm of 11-Amino-11-N-[4-[4-(3-pyridyl) imidazol-1-yl]butyl-11-deoxy-3-oxo-5-O-desosaminyl-6-O-methylerythronolide A 11,12-cyclic carbamate [Telithromycin](1).

### Example 13

### 11-Amino-11-N-[4-[4-(3-pyridyl)imidazol-1-yl]butyl-11-deoxy-3-oxo-5-O-desosaminyl-6-O-methylerythronolide A 11,12-cyclic carbamate [Telithromycin] (Formula I)

N-Chloro succinimide (4.68 gm) was charged to the reaction vessel under nitrogen atmosphere and Dichloromethane (200 ml) was added slowly. The reaction masses was cooled to 0° C., Dimethyl sulfide (3.5 ml) was added slowly and continue the stirring at same temperature for half an hour. The reaction mass was cooled to −25° C. and 2′-hydroxy-11-amino-11-N-[4-[4-(3-pyridyl)imidazol-1-yl]butyl]-11-deoxy-5-O-desosaminyl-6-O-methylerythronolide A 11,12-cyclic carbamate (10 g) dissolved in 50 ml dichloromethane was added slowly. The mixture was stirred at −25° C. temperature for 2 hour. At the same temperature Diisopropylethyl amine (0.6 ml) was added and the reaction mixture was stirred for half an hour. Water (500 ml) was added and stirred the mixture for 30 min. The organic layer was separated and washed with water, dried over sodium sulfate and distilled off the solvent under vacuum to give 6.5 gm of 11-Amino-11-N-[4-[4-(3-pyridyl)imidazol-1-yl]butyl-11-deoxy-3-oxo-5-O-desosaminyl-6-O-methylerythronolide A 11,12-cyclic carbamate [Telithromycin] (Formula I).

### Example 14

### Preparation of 2′,4″-di-O-benzoyl-6-O-methylerythromycin A (IXa)

1250 ml of ethyl acetate was added to 250 gm Clarithromycin A. 264.65 g benzoic anhydride, 57.20 g 4-dimethylamino pyridine and 67.60 g tri ethyl amine was added to the reaction mixture at 25° C. to 35° C. The reaction mixture was stirred for about 70 hours at ambient temperature After the completion of reaction, ethyl acetate was distilled out to obtain 2′,4″-di-O-benzoyl-6-O-methylerythromycin A

### Example 15

### Preparation of 10,11-anhydro-2′,4″-di-O-benzoyl-12-O-imidazolylcarbonyl-6-O-methylerythromycin A (Xa)

1000 ml Dimethylformamide is added to 2′,4″-di-O-benzoyl-6-O-methylerythromycin A at 25° C. to 35° C. 63.70 g DBU (1,8-Diazabicyclo[5.4.0]undec-7-ene) was added to the reaction mixture and stirred at ambient temperature. Further, 170 g 1,1-Carbonyl diimidazole was added to the reaction mass and it was stirred until completion of reaction at ambient temperature. The desired compound is isolated by addition of water

### Example 16

### Preparation of 2′,4″-di-O-benzoyl-11-amino-11-N-[4-[4-(3-pyridyl)imidazol-1-yl]butyl]-11-deoxy-6-O-methylerythromycin A 11,12-cyclic carbamate (XIa)

1000 ml dimethylformamide was added to 200 g 10,11-anhydro-2′,4″-di-O-benzoyl-12-O-imidazolylcarbonyl-6-O-methylerythromycin A at 25° C. to 35° C. 63 g of 4-[4-(3-pyridyl)imidazol-1-yl]butylamine and 29.50 g DBU was added to the reaction mixture and it was stirred at 25° C. to 35° C. until the completion of reaction. It is further treated with cold water and obtained solid was treated with dichloromethane followed by extraction and removal of solvent to give 2′,4″-di-O-benzoyl-11-amino-1-N-[4-[4-(3-pyridyl)imidazol-1-yl]butyl]-11-deoxy-6-O-methylerythromycin A 11,12-cyclic carbamate.

### Example 17

### Preparation of 2′-O-benzoyl-11-amino-11-N-[4-[4-(3-pyridyl)imidazol-1-yl]butyl]-11-deoxy-5-O-desosaminyl-6-O-methylerythronolide A 11,12-cyclic carbamate (XIIa)

400 ml acetone was added to 200 g 2′,4″-di-O-benzoyl-11-amino-11-N-[4-[4-(3-pyridyl)imidazol-1-yl]butyl]-11-deoxy-6-O-methylerythromycin A 11,12-cyclic carbamate to obtain clear solution at 25° C. to 35° C. Dilute hydrochloric acid (400 ml) was added to the reaction mixture and it was stirred for 24 hours at ambient temperature. After the completion of the reaction, the reaction mixture was extracted with ethyl acetate and treated with Sodium hydroxide solution to give 2′-O-benzoyl-11-amino-11-N-[4-[4-(3-pyridyl)imidazol-1-yl]butyl]-11-deoxy-5-O-desosaminyl-6-O-methylerythronolide A 11,12-cyclic carbamate (XIIa)

### Example 18

### Preparation of 2′-benzoyl-11-amino-11-N-[4-[4-(3-pyridyl)imidazol-1-yl]butyl]-11-deoxy-5-O-desosaminyl-6-O-methylerythronolide A 11,12-cyclic carbamate (XIIIa)

180 ml dichloromethane was added to 8.0 g N-chlorosuccinimide under nitrogen at room temperature cooled to 0° C. 7.2 ml dimethylsulfide was added slowly to the reaction mixture at 0° C. under stirring. 20 g 2′-O-benzoyl-11-amino-11-N-[4-[4-(3-pyridyl)imidazol-1-yl]butyl]-11-deoxy-5-O-desosaminyl-6-O-methylerythronolide A 11,12-cyclic carbamate dissolved in 80 ml dichloromethane was added drop wise to the reaction mixture at 0° C. under stirring. Further it was cooled to about −20° C. and solution of 16 ml Triethylamine in 20 ml dichloromethane is added to the reaction mixture and stirred for 30 minutes. After completion of the reaction, it is treated with saturated sodium bicarbonate solution and organic layer separated out. Desired product is obtained from by distillation of solvent from organic layer

### Example 19

### Preparation of 11,12-dideoxy-3-de[(2,6-dideoxy-3-C-methyl-3-O-methyl-alpha-L-ribohexopyranosyl)oxy]-6-O-methyl-3-oxo-12,11-(oxycarbonyl[4-[4-(3-pyridinyl)-1H-imidazol-1-yl]butyl]imino)-erythromycin. (Telithromycin)

100 ml methanol was added to 10 g 2′-benzoyl-11-amino-11-N-[4-[4-(3-pyridyl)imidazol-1-yl]butyl]-11-deoxy-5-O-desosaminyl-6-O-methylerythronolide A 11,12-cyclic carbamate at 25° C. to 30° C. and the reaction mixture was heated to reflux for about 7 hours. After completion of the reaction, Methanol was distilled off under vacuum at 45° C. 100 ml dilute hydrochloric acid was added to the residue and the aqueous layer was extracted with ethyl acetate (3×40 ml) and sodium bicarbonate and organic layer separated out. The product is obtained by distillation of solvent from organic layer and recrystallized from Methyl tert-butyl ether (MTBE) and cyclohexanone to give Telithromycin.

While the present invention has been described in terms of its specific embodiments, certain modifications and equivalents will be apparent to those skilled in the art and are intended to be included within the scope of the present invention.

1. A process for the preparation of compound of formula (I) (Telithromycin) or its pharmaceutically acceptable salts 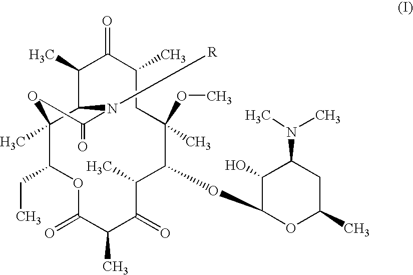
where, R is 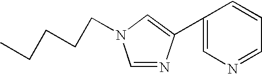
the process comprising the steps of
(a) reacting compound of formula (IX) 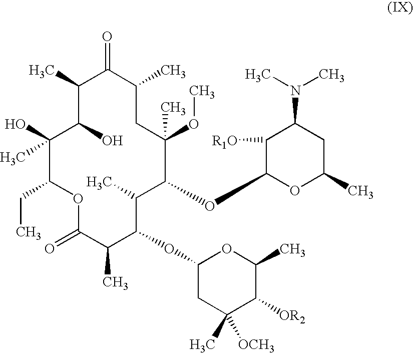
with carbonyldiimidazole in presence of a polar solvent and base to obtain the compound of formula (X) 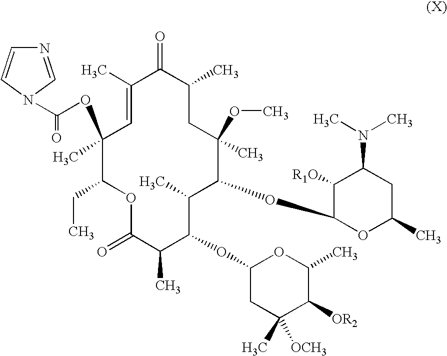
where R₁ and R₂ are same or different protecting groups represented by 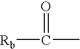
R_{b} is C₁ to C₁₀ alkyl group or aryl group, preferably R_{b} is C₁-C₄ alkyl group, aryl represents substituted or unsubstituted phenyl group; more preferably R₁ and R₂ are same or different selected from acetyl, benzyl or benzoyl group
(b) condensing the compounds of formula (X) with R—NH₂ in a suitable polar solvent to obtain compounds of formula (XI) 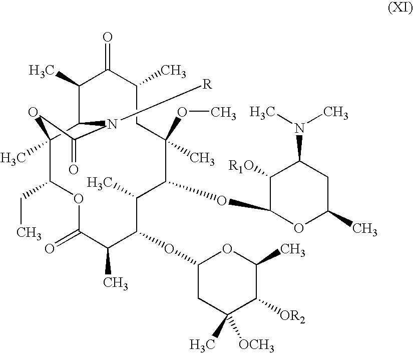
where R is as defined above and R₁ and R₂ are same or different protecting groups as described above;
(c) treating the obtained compound formula (XI) with an acid to give the compound of formula (XII) 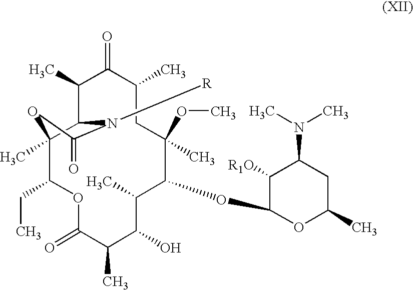
(d) oxidizing the resulting compounds of formula (XII) in presence of oxidizing agent to formula (XIII) 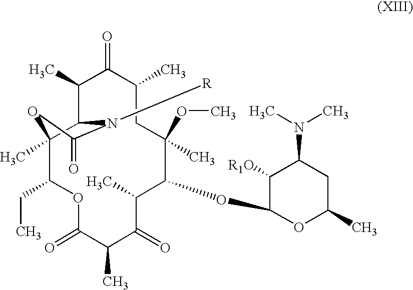
(e) removing the protecting group at 2′ position of formula (XIII) by treating with an alcohol to give Telithromycin of Formula (I)

2. A process for the preparation of compounds of formula (I) or its pharmaceutically acceptable salts 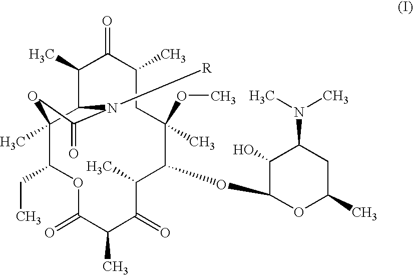
where, R is 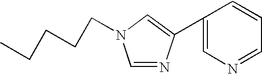
the process comprising the steps of
(a) reacting compound of formula (IX) 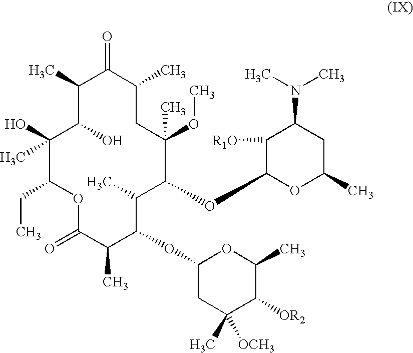
with carbonyldiimidazole in presence of a polar solvent and base to obtain the compound of formula (X), 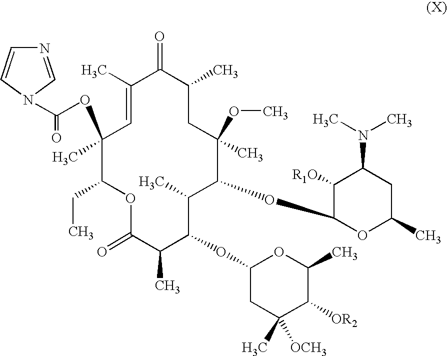
where R₁ and R₂ are same or different protecting groups represented by 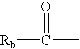
R_{b} is C₁ to C₁₀ alkyl group or aryl group, preferably R_{b} is C₁-C₄ alkyl group, aryl represents substituted or unsubstituted phenyl group, more preferably R₁ and R₂ are same or different selected from acetyl, benzyl or benzoyl group;
(b) condensing the compounds of formula (X) with R—NH₂ in a suitable polar solvent to obtain compounds of formula (XI) 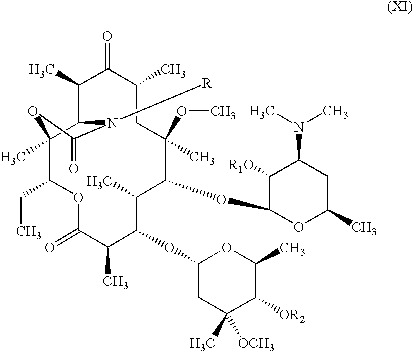
where R is as defined above and R₁ and R₂ are same or different protecting groups as described above;
(c) treating the obtained compound formula (XI) with an acid to give compound of formula (XII) 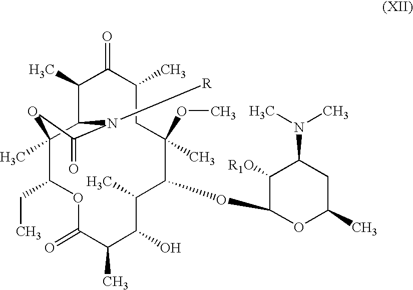
(d) treating compounds of formula (XII) with an alcohol to give compounds of formula (XIV) 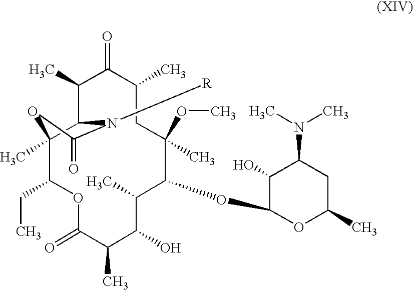
(e) oxidizing the resulting compounds of formula (XIV) of step (d) selectively in presence of oxidizing agent to obtain Telithromycin formula (I).

3. A process as claimed in claim 1, wherein said polar solvent in step (a) is selected from dimethylformamide, tetrahydrofuran, acetonitrile and mixtures thereof.

4. A process as claimed in claim 1, wherein said base in step (a) is selected from DBU, triethylamine, diisopropylethylamine.

5. A process as claimed in claim 1, wherein said polar solvent in step (b) is selected from group comprising of methanol, ethanol, isopropanol, n-propanol, n-butanol, iso butyl alcohol, tert-butyl alcohol, methoxyethanol, ethoxyethanol, pentanol, neo-pentyl alcohol, tert-pentyl alcohol, cyclohexanol, ethylene glycol, propylene glycol, benzyl alcohol, phenol, glycerol, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethylsulfoxide, propionitrile, ethyl formate, methyl acetate, hexachloroacetone, HMPA, HMPT, acetone, ethyl methyl ketone, ethyl acetate, isopropyl acetate, t-butyl acetate, sulfolane, N,N-dimethylpropionamide, nitromethane, nitrobenzene, tetrahydrofuran (THF), dioxane, water, polyethers or mixtures thereof.

6. A process as claimed in claim 5, wherein said polar solvent is selected from dimethylformamide or acetonitrile.

7. A process as claimed in claim 1, wherein said step (b) is carried out in presence or absence of base selected from DBU, triethylamine, diisopropylethylamine

8. A process as claimed in claim 1, wherein said step (b) is carried out at a temperature 5° C. to 120° C.

9. A process as claimed in claim 8, wherein the said step (b) is carried out preferably at a temperature 30° C. to 60° C.

10. A process as claimed in claim 1, wherein the said acid in step (c) is selected from organic or inorganic acid.

11. A process as claimed in claim 10, wherein the said acid is selected from the group comprising of hydrochloric acid, hydrobromic acid, sulfuric acid, nitric acid, phosphoric acid, perchloric acid or hydrofluoric acid.

12. A process as claimed in claim 11, wherein the acid is preferably hydrochloric acid.

13. A process as claimed in claim 1, wherein step (c) is carried out in a solvent selected from water, polar organic solvents or mixtures thereof.

14. A process as claimed in claim 13, wherein said solvent is selected from water, alcohol or mixtures thereof.

15. A process as claimed in claim 14, wherein said solvent is selected from water, methanol, ethanol, isopropanol, n-propanol, tert-butanol, n-butanol or mixtures thereof.

16. A process as claimed in claim 1, wherein said step (c) is carried out at a temperature 0° C. to 70° C.

17. A process as claimed in claim 16, where in step (c) is carried out at a temperature 20° C. to 60° C.

18. A process as claimed in claim 1, wherein oxidation said in step (d) is carried out using Corey-Kim oxidation method, Dess-Martin reagent, Pfitzner Moffat method or modifications thereof or with dimethyl sulfoxide in presence of oxalyl chloride or phosphorous pentoxide or p-Toluene sulfonyl chloride or acetic anhydride or N-chlorosuccinimide or by manganese or chromium or selenium reagents, tert-amine oxides or any said oxidant in presence or absence of phase transfer catalyst.

19. A process as claimed in claim 1, wherein alcohol said in step (e) is selected from group comprising of methanol, ethanol, n-propanol, iso propanol, tert-butanol, n-butanol or mixtures thereof.

20. A process as claimed in claim 19, wherein the said alcohol is preferably methanol.

21. A process as claimed in claim 1, wherein said step (e) is carried out in presence or absence of mineral acid selected from HCl, H₂SO₄

22. A process as claimed in claim 1, wherein said step (e) is carried out at a temperature of 0° C. to 100° C.

23. A process as claimed in claim 22, wherein step (e) is carried out preferably at a temperature of 20° C. to 70° C.

24. A process as claimed in claim 2, wherein said alcohol in step (d) is selected from group comprising of methanol, ethanol, n-propanol, iso propanol, tert-butanol, n-butanol or mixtures thereof.

25. A process as claimed in claim 24, wherein said alcohol is preferably methanol.

26. A process as claimed in claim 2, wherein said step (d) is carried out at a temperature of 0 to 70° C.

27. A process as claimed in claim 26, wherein the temperature is between 20 to 65° C.

28. A process as claimed in claim 2, wherein said oxidation in step (e) is carried out using Corey-Kim oxidation method, Des-Martins reagent, Pfitzner moffat method or modifications thereof or with dimethyl sulfoxide in presence of oxalyl chloride or phosphorous pentoxide or p-Toluene sulfonyl chloride or acetic anhydride.

29. A process as claimed in claim 2, wherein oxidation in step (e) is carried out by manganese or chromium or selenium reagents, tert-amine oxides or any above oxidant in presence of phase transfer catalyst.

30. The novel compounds
(i) 10,11-Anhydro-2′,4″-di-O-benzoyl-12-O-imidazolylcarbonyl-6-O-methylerythromycin A of formula (Xa) 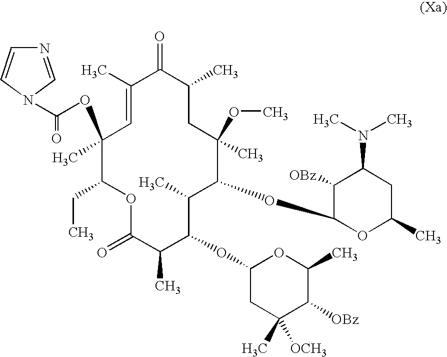
(ii) 2′,4″-di-O-benzoyl-11-amino-11-N-[4-[4-(3-pyridyl)imidazol-1-yl]butyl]-11-deoxy-6-O-methylerythromycin A 11,12-cyclic carbamate of formula (XIa) 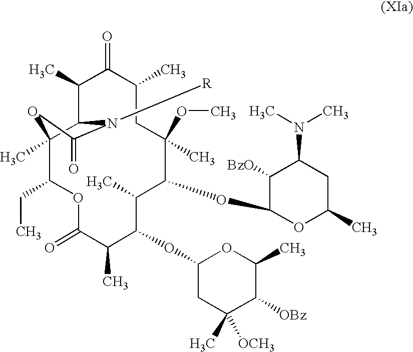
(iii) 2′-O-benzoyl-11-amino-11-N-[4-[4-(3-pyridyl)imidazol-1-yl]butyl]-11-deoxy-5-O-desosaminyl-6-O-methylerythronolide A 11,12-cyclic carbamate of formula (XIIa) 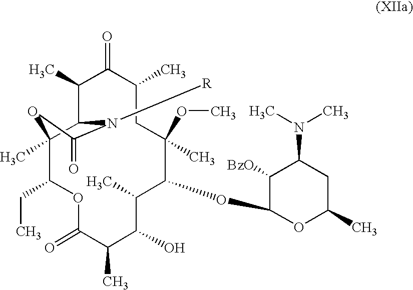
(iv) 2′-benzoyl-11-amino-11-N-[4-[4-(3-pyridyl)imidazol-1-yl]butyl]-11-deoxy-5-O-desosaminyl-6-O-methylerythronolide A 11,12-cyclic carbamate of formula (XIIa) 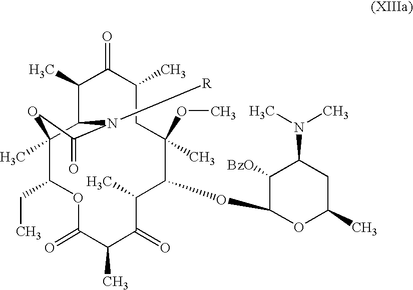
where R is

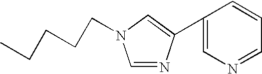

31. A process for the preparation of compound of formula (XIIIa) 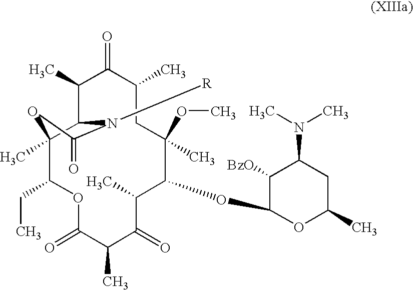
where, R is 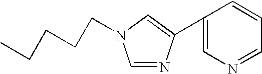
the process comprising the steps of
(a′) reacting 2′,4″-di-O-benzoyl-6-O-methylerythromycin A compound of formula (IXa) 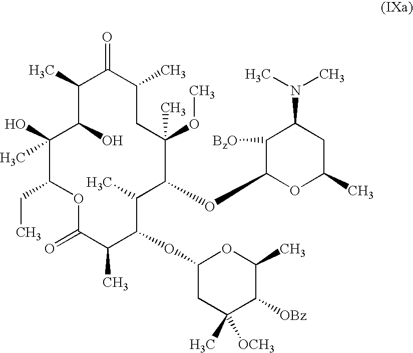
with carbonyldiimidazole in presence of a polar solvent and base to obtain 10,11-anhydro-2′,4″-di-O-benzoyl-12-O-imidazolylcarbonyl-6-O-methylerythromycin A (Xa) 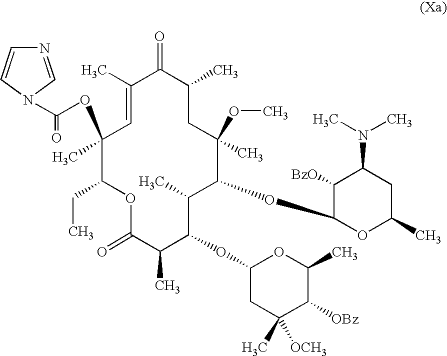
(b′) condensing the compounds of formula (Xa) with R—NH₂ in a suitable polar solvent in presence of base to obtain 2′,4″-di-O-benzoyl-11-amino-11-N-[4-[4-(3-pyridyl)imidazol-1-yl]butyl]-11-deoxy-6-O-methylerythromycin A 11,12-cyclic carbamate of formula (XIa) 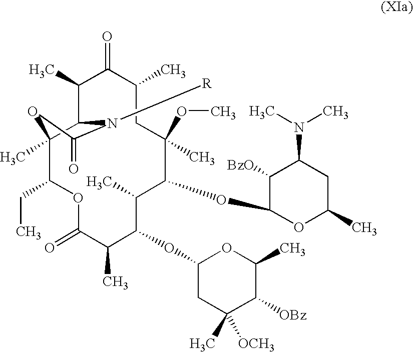
where R is as defined above
(c′) treating the obtained compound formula (XIa) with an acid to give 2′-O-benzoyl-11-amino-11-N-[4-[4-(3-pyridyl)imidazol-1-yl]butyl]-11-deoxy-5-O-desosaminyl-6-O-methylerythronolide A 11,12-cyclic carbamate of formula (XIIa) 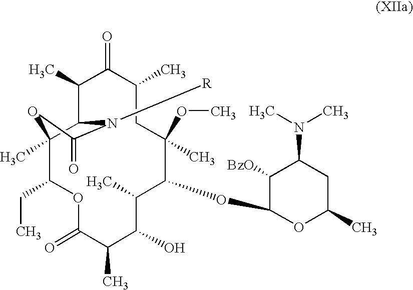
(d′) oxidizing the resulting compounds of formula (XIIa) in presence of oxidizing agent to obtain the compound of formula (XIIIa)

32. A process as claimed in claim 31, wherein said polar solvent in step (a′) is selected from dimethylformamide, tetrahydrofuran, acetonitrile and mixtures thereof.

33. A process as claimed in claim 31, wherein said base in step (a)′ is selected from DBU, triethylamine, diisopropylethylamine.

34. A process as claimed in claim 31, wherein said polar solvent in step (b′) is selected from group comprising of methanol, ethanol, isopropanol, n-propanol, n-butanol, iso butyl alcohol, tert-butyl alcohol, methoxyethanol, ethoxyethanol, pentanol, neo-pentyl alcohol, tert-pentyl alcohol, cyclohexanol, ethylene glycol, propylene glycol, benzyl alcohol, phenol, glycerol, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethylsulfoxide, propionitrile, ethyl formate, methyl acetate, hexachloroacetone, HMPA, HMPT, acetone, ethyl methyl ketone, ethyl acetate, isopropyl acetate, t-butyl acetate, sulfolane, N,N-dimethylpropionamide, nitromethane, nitrobenzene, tetrahydrofuran (THF), dioxane, water, polyethers or mixtures thereof.

35. A process as claimed in claim 31, wherein said polar solvent is selected from dimethylformamide or acetonitrile.

36. A process as claimed in claim 31, wherein base said in step (b′) is selected from DBU, triethylamine, diisopropylethylamine.

37. A process as claimed in claim 31, wherein the said step (b′) is carried out preferably at a temperature 30° C. to 60° C.

38. A process as claimed in claim 31, wherein the said acid in step (c′) is selected from organic or inorganic acid selected from the group comprising of hydrochloric acid, hydrobromic acid, sulfuric acid, nitric acid, phosphoric acid, perchloric acid or hydrofluoric acid.

39. A process as claimed in claim 31, wherein step (c′) is carried out in a solvent selected from water, methanol, ethanol, isopropanol, n-propanol, tert-butanol, n-butanol or mixtures thereof.

40. A process as claimed in claim 31, where in step (c′) is carried out at a temperature 20° C. to 60° C.

41. A process as claimed in claim 31, wherein oxidation said in step (d′) is carried out using Corey-Kim oxidation method, Dess-Martin reagent, Pfitzner Moffat method or modifications thereof or with dimethyl sulfoxide in presence of oxalyl chloride or phosphorous pentoxide or p-Toluene sulfonyl chloride or acetic anhydride or N-chlorosuccinimide by manganese or chromium or selenium reagents, tert-amine oxides or any said oxidant in presence of phase transfer catalyst.

42. Use of compounds of formula (Xa), (XIa), (XIIa), (XIIIa) for the preparation of Telithromycin (I)
